(19) United States
(12) Patent Application Publication
(10) Pub. No.: US 2025/0018005 A1
(43) Pub. Date: Jan. 16, 2025

(54) ANTI-FUNGAL GRIFFITHSIN COMPOSITIONS AND METHODS OF USE

(71) Applicant: University of Louisville Research Foundation, Inc., Louisville, KY (US)

(72) Inventors: Henry Nabeta, Louisville, KY (US); Joseph Kouokam, Louisville, KY (US); Kenneth E. Palmer, Louisville, KY (US)

(21) Appl. No.: 18/290,872

(22) PCT Filed: Jul. 28, 2022
(86) PCT No.: PCT/US2022/074248

§ 371 Date: Jan. 22, 2024

Related U.S. Application Data
(60) Provisional application No. 63/226,234, filed on Jul. 28, 2021.

(51) Int. Cl. A61K 38/16; A61P 31/10

(52) U.S. Cl. CPC A61K 38/16; A61P 31/10

(57) ABSTRACT

Anti-fungal compositions based on the griffithsin protein and methods of using the same have been identified. The compositions display antifungal activity against species of Candida fungus.

### CROSS-REFERENCE TO RELATED APPLICATION

This Application claims the benefit of U.S. provisional patent application Ser. No. 63/226,234, filed 28 Jul. 2021, for ANTI-FUNGAL GRIFFITHSIN COMPOSITIONS AND METHODS OF USE, incorporated herein by reference.

### GOVERNMENT RIGHTS

This invention was made with government support under grant numbers U01HL127518 and 5U19AI113182 awarded by the National Institutes of Health. The government has certain rights in the invention.

### FIELD OF THE INVENTION

Anti-fungal compositions based on the griffithsin protein and methods of using the same have been identified. The compositions display antifungal activity against species of Candida fungus.

### BACKGROUND OF THE INVENTION

Candidiasis is a fungal infection caused by a fungus of the genus Candida. Vulvovaginal candidiasis (VVC) infection results in itching, discharge, soreness, burning sensation and may lead to significant distress among individuals. Candida organisms are commensals in the vagina, with overgrowth resulting in vaginal and vulval inflammation contributing to the pathological hallmarks of infection. While the immune system is typically able to clear the infection, many women suffer from prolonged episodes of infection, with others reporting recurrent infections. Long term use of anti-fungal drugs such as azoles, echinocandins and polyenes, recurrent mucosal infections including chronic mucocutaneous candidiasis in human immunodeficiency virus (HIV) infection and suboptimal infection control have led to increasing incidence of anti-fungal drug resistance. In particular there are increasing reports of Candida species that demonstrate high-resistance to fluconazole and other antifungal drugs. Difficulty in managing therapy of patients with multi drug-resistant Candida species, and pan-resistant isolates of Candida auris in the U.S. and globally has further highlighted an urgent need to develop new antifungal agents.

Lectins are proteins that possess the ability to bind carbohydrates, often with high affinity and specificity. Griffithsin (GRFT) is an antiviral lectin originally derived from the red alga Griffithsia sp. and has been widely studied for its potent and broad-spectrum activity against human immunodeficiency virus (HIV) and other viruses. Native GRFT, a domain-swapped homodimer, binds to glycoproteins in the viral envelope and outer structure in a monosaccharide-dependent manner. Additionally, antiviral properties of GRFT are based on efficient and effective binding to oligosaccharides with mannose terminal-branches, as well as N-acetylglucosamine and glucose, with lower affinity compared with mannose.

### SUMMARY

Disclosed herein is Q-Griffithsin (Q-GRFT), a recombinant oxidation-resistant variant of GRFT, and methods of using the same as an antifungal agent. A surface methionine residue at amino acid position 78 of wild-type GRFT was found to be susceptible to oxidation and may therefore have deleterious effect on protein stability. The residue was replaced with a glutamine (Q) residue, and this recombinant protein was found to have superior environmental stability and similar antiviral activity to wild-type GRFT. GRFT is a polypeptide comprising the amino acid sequence of: (SEQ ID NO: 1) SLTHRKFGGSGGSPFSGLSSIAVRSGSYLDAIIIDGVHHGGSGGNLSPTFTFGSGEY ISNMTIRSGDYIDNISFETNMGRRFGPYGGSGGSANTLSNVKVIQINGSAGDYLDSL D IYYEQY. Q-GRFT is a polypeptide comprising the amino acid sequence of: (SEQ ID NO: 2) SLTHRKFGGSGGSPFSGLSSIAVRSGSYLDAIIIDGVHHGGSGGNLSPTFTFGSGEY ISNMTIRSGDYIDNISFETNQGRRFGPYGGSGGSANTLSNVKVIQINGSAGDYLDSL D IYYEQY, with the modified residue 78 indicated by underline.

Q-GRFT was determined to possess hitherto unreported antifungal activity, making this recombinant protein a candidate antifungal agent. Disclosed herein are findings of the impact of Q-GRFT on growth of Candida species and identification of a possible mechanism of antifungal activity. More specifically, Q-GRFT has been demonstrated to bind to α-mannan in the Candida albicans cell wall. Q-GRFT binding was found to disrupt cell wall integrity and induced reactive oxidative species (ROS) formation, resulting in cell death. Q-GRFT also inhibited growth of other Candida species, including C. glabrata, C. parapsilosis and C. krusei, with modest activity against some strains of multi- and pan-resistant C. auris. Q-GRFT induced differential expression of numerous genes involved in response to cell stress including those responsible for neutralizing ROS production and cell cycle regulation.

Furthermore, the efficacy of Q-GRFT was evaluated in prophylactic and therapeutic murine models of VVC. In a preventive model, in comparison with infected controls, Q-GRFT treatment resulted in a lower fungal burden but did not alter the number of vaginal neutrophils and monocytes. In a therapeutic model, Q-GRFT enhanced fungal clearance when compared with infected untreated controls. Finally, histopathology demonstrated lower vaginal colonization with C. albicans following Q-GRFT treatment. This novel anti-fungal activity indicates that Q-GRFT may be useful in methods of prevention and treatment of candidiasis, and VVC in particular, as a topical product or via other delivery methods.

In some embodiments, the present invention comprises a method for preventing or treating fungal infections, including applying a composition including griffithsin protein to a subject. In further embodiments, the fungal infection is vaginal candidiasis and the subject is a subject vagina. In certain embodiments, the griffithsin protein is a mutant griffithsin protein, such as Q-GRFT. In further embodiments, the composition includes griffithsin protein at a concentration effective to reduce fungal infection. In some embodiments, the griffithsin protein is present at a concentration of about 1% by weight, a non-zero concentration of not more than 1% by weight, a concentration of 0.001% to 0.5% by weight, a concentration of 0.01% to 0.3% by weight, a concentration of at least 1 μg/mL, a concentration between 1 μg/mL and 10 mg/mL (7.8 μM), a non-zero concentration of less than 10 mg/mL (7.8 μM), or a concentration between 6 μg/mL and 95 μg/mL. In further embodiments, the composition further includes a carrier, such as a biologically-compatible suspending agent, such as a carbomer gel. In certain embodiments, applying the composition includes applying the composition topically. In further embodiments, applying the composition includes applying the composition topically to a subject vagina. In some embodiments, applying the composition includes applying the composition at least twice per day for a period of at least three days, a period of three days to ten days, a period of least four days, a period of four days to eight days, or a period of five days to seven days. In further embodiments, applying the composition includes applying the composition until the fungal infection is no longer detectable. In certain embodiments, applying the composition includes applying the composition at a volume per application in the range of 0.1 mL to 10 mL, in the range of 0.1 mL to 5.0 mL, in the range of 0.1 mL to 2.0 mL, or in the range of 0.5 mL to 1.0 mL.

In some embodiments, the present invention comprises an antifungal composition comprising a griffithsin protein and a carrier. In further embodiments, the griffithsin protein is a mutant griffithsin protein, such as Q-GRFT. In certain embodiments, the griffithsin protein is present at a concentration effective to reduce fungal infection. In some embodiments, the griffithsin protein is present at a concentration of about 1% by weight, a non-zero concentration of not more than 1% by weight, a concentration of 0.001% to 0.5% by weight, a concentration of 0.01% to 0.3% by weight, a concentration of at least 1 μg/mL, a concentration between 1 μg/mL and 10 mg/mL (7.8 μM), a non-zero concentration of less than 10 mg/mL (7.8 μM), or a concentration between 6 μg/mL and 95 μg/mL. In some embodiments, the fungal infection is candidiasis. In further embodiments, carrier is a biologically-compatible suspending agent, such as a carbomer gel.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 is a pair of graphs depicting the effect of Q-GRFT on the growth of Candida albicans: (A) Candida albicans ATCC 32032 at a concentration of 1.0×10⁵ cell/mL was incubated with varying concentrations of Q-GRFT and fluconazole control at 37° C. in Sabourand Dextrose culture medium and growth monitored at 24, 48 and 72 hours. (B) C. albicans (1.0×10⁵ cells/mL) was incubated with varying concentrations of Q-GRFT^{lec neg} (7.80 M, 0.78 M), with fluconazole (3.3 mM) and PBS as controls, at 37° C. in Sabourand Dextrose culture medium. Growth was monitored at 24 and 48 hours. Fungal counts were performed using either a Bio-Rad TC10™ Automated cell counter, Singapore, or an ECHO Rebel hybrid microscope (RBLTEW31), San Diego, USA. Both experiments were performed in triplicate. Representative data (mean±SD) from at least 3 independent experiments is shown.

FIG. 2 illustrates QGRFT binding to Candida albicans. Panel A is a graph depicting fluorescence intensity mean±SD of Candida albicans cultured with either Fluorescein-Q-GRFT, Q-GRFT or Q-GRFT^{lec neg}, at lectin concentrations of 7.8 μM. Panel B is a series of fluorescence microscopy images following culture of Candida albicans with Fluorescein-Q-GRFT. Green fluorescence demonstrates localization to C. albicans cells. DAPI demonstrates DNA staining. Scale bars are 3 μm. Experiments were performed in triplicate.

FIG. 3 is a series of graphs depicting Q-GRFT binding to Candida albicans cell wall components. (A) Q-GRFT binds to Candida albicans cell wall component α-mannan but not chitin or β-glucan. (B) Unlike the low concentration (0.005 μg/mL), higher concentrations of α-mannan (0.5 g/mL and 0.05 μg/mL) inhibited Q-GRFT's binding to gp120. Q-GRFT's ability to bind to cell wall components of Candida albicans was determined using an ELISA binding assay. A competition ELISA assay was performed to determine Q-GFRT binding to gp120 in the presence of a-mannan. Experiments were performed in triplicate and repeated at least 3 times.

FIG. 4 illustrates the effects of Q-GRFT on Candida albicans' cell integrity and oxidative status. (A) Yeast cells were incubated in the absence or presence of Q-GRFT (7.8 μM) overnight at 37° C., followed by staining with Trypan Blue to detect non-viable cells. (B) Candida albicans cells were incubated with Q-GRFT (7.8 μM) or PBS overnight followed by incubation in the presence of 0.5 mg/mL DAB for 2 hours to detect hydrogen peroxide. Quantification of the proportion (percentage) of DAB staining cells following either Q-GRFT or PBS treatment for the experiments is shown. (C) Candida albicans was incubated overnight with Q-GRFT (7.8 and 0.78 μM) or medium only at 37° C. Cells were harvested and 3.2×10⁶ cells per treatment used for direct fluorescence using the H2DCF-DA assay to determine Reactive Oxygen Species (ROS) levels in the cells. Experiments were performed in triplicate.

FIG. 5 illustrates C. albicans surface phenotype following treatment with Q-GRFT. C. albicans was grown overnight in Sabourand Dextrose medium in presence of either PBS vehicle (A-C) or 7.8 μM Q-GRFT (D-E) at 37° C. Cells were then observed by SEM. (A) Budding morphology of C. albicans. Note the spherical to oval appearance of cells. Magnification×7500. (B) Cells with smooth surface, polar budding and bud scars (arrows). Magnification×10000. (C) Note the smooth cell surface and absence of any ‘non-polar’ bud scars. Magnification×12500. (D) Cells with spherical to circular shape with rough surface. Magnification×7500. (E) Note the rough surface, uniform indentations with a desiccated and wrinkled appearance. Magnification×10000. (F) Note the loss of polar budding, more circular appearance, and cells with multiple bud scars (arrows) upon treatment with Q-GRFT. Magnification×17500. Spherical to oval appearance and loss of polar budding indicate fungal cells dying upon treatment with Q-GRFT.

FIG. 6A illustrates the impact of Q-GRFT on growth of non-Candida albicans species at 24, 48 and 72 hours in (A) Candida glabrata CDC316, (B) Candida krusei CDC397, and (C) Candida parapsilosis CDC337.

FIG. 6B illustrates the impact of Q-GRFT on growth of non-Candida albicans species at 24, 48 and 72 hours in multi-drug resistant Candida auris strains (D) Candida auris CDC389 (E) Candida auris CDC389 (F) Candida auris CDC383.

FIG. 6C illustrates the impact of Q-GRFT on growth of non-Candida albicans species at 24, 48 and 72 hours in multi-drug resistant Candida auris strains (G) Candida auris CDC384 (H) Candida auris CDC385 and (I) Candida auris CDC386.

FIG. 7A illustrates a murine model for evaluating the effect of Q-GRFT on vaginal candidiasis. The model is performed using 6-8 weeks old CBA/J mice. Inoculum: 1×10⁸ cells/ml, 20 μL.

FIG. 7B is a series of plots depicting murine vaginal C. albicans burden pre-treatment (top row) and post-treatment (bottom row) with Q-GRFT solution as compared to PBS (left column), Q-GRFT gel as compared to placebo gel (middle column), and Nystatin, a known anti-fungal medication, as compared to PBS (right column).

FIG. 7C are photographs of plates comparing an infected control and and cells treated with Q-GRFT solution.

FIG. 8A depicts representative flow cytometry plots of granulocytes CD45+, neutrophils (CD45+, Ly6G+, CD11b+).

FIG. 8B is a series of plots depicting murine vaginal granulocytes (top row), vaginal neutrophils (middle row), and vaginal monocytes (bottom row) post-treatment with Q-GRFT solution as compared to PBS (left column), Q-GRFT gel as compared to placebo gel (middle column), and Nystatin, a known anti-fungal medication, as compared to PBS (right column).

FIG. 9 is a series of vaginal tissue histology images (A) hematoxylin and eosin (H&E) stained, PBS-treated, (B) Periodic acid-Schiff (PAS) stained, PDB-treated, (C) a close-up image of panel (B), (D) H&E stained, Q-GRFT-treated, (E) PAS stained, Q-GRFT-treated, (F) a close-up image of panel (E).

FIG. 10A is a schematic depicting the timeline of an experimental model of vaginal infection to evaluate the efficacy of Q-GRFT in a preventative murine model. CBA/J mice (N=10 per group) were estradiol-treated at Day-3, followed by twice daily instillation of either Q-GRFT gel, nystatin solution, PBS, or carbomer gel (such as, for example Carbopol®) placebo per vaginum for the next 5 days. At Day 0, mice were inoculated with 20 μL of C. albicans blastospores at a cell concentration of 1.0×10⁸ CFU/mL, per vaginum. A vaginal lavage was performed 24 hours after the final dose administration.

FIG. 10B is a graph depicting vaginal fungal burden (CFU/mL) following treatment, for mice treated according to FIG. 11A. Each dot represents one mouse, N=10 mice per group. Experiments were performed and repeated at least 2 times, and representative data of Mean±SEM is shown.

FIG. 10C depicts flow cytometry plots of neutrophils (upper panel) and mononuclear phagocytes (lower panel) in the vaginal lavage. Neutrophils were identified as CD45⁺Ly6G⁺CD11b⁺, while mononuclear phagocytes were CD45⁺CD11⁺F4/80⁺ cells.

FIG. 10D is a graph depicting neutrophil cell populations in the vaginal lavage following treatment. N=10 animals per group, and each dot represents a population of cells from a single mouse. Measurements are representative of cell populations from experiments performed at least 2 times. Mean±SEM data is presented.

FIG. 10E is a graph depicting mononuclear phagocyte cell populations in the vaginal lavage following treatment. N=10 animals per group, and each dot represents a population of cells from a single mouse. Measurements are representative of cell populations from experiments performed at least 2 times. Mean±SEM data is presented.

FIG. 11A is a schematic depicting the timeline of an experimental model of vaginal infection to evaluate the efficacy of Q-GRFT in a therapeutic murine model. CBA/J mice (N=20 per group) were estradiol-treated at Day-3. At Day 0, mice were inoculated with 20 μL of C. albicans blastospores at a cell concentration of 1.0×10⁸ CFU/mL, per vaginum. Treatment with Q-GRFT gel, nystatin solution, PBS, or carbomer placebo gel began on Day 5 and continued twice daily for 7 days. Vaginal lavage was performed on Day 4 and Day 12 to establish pre-treatment and post-treatment fungal burder.

FIG. 11B is a graph depicting vaginal fungal burden (CFU/mL) on Day 4 (pre-treatment), for mice treated according to FIG. 12A. Each dot represents one mouse, N=20 mice per group. Experiments were performed and repeated at least 2 times, and representative data of Mean±SEM is shown.

FIG. 11C is a graph depicting vaginal fungal burden (CFU/mL) on Day 12 (post-treatment). Each dot represents one mouse, N=20 mice per group. Experiments were performed and repeated at least 2 times, and representative data of Mean±SEM is shown.

FIG. 11D is a graph depicting neutrophil cell populations in the vaginal lavage following treatment. N=20 animals per group, and each dot represents a population of cells from a single mouse. Measurements are representative of cell populations from experiments performed at least 2 times. Mean±SEM data is presented.

FIG. 11E is a graph depicting mononuclear phagocyte cell populations in the vaginal lavage following treatment. N=20 animals per group, and each dot represents a population of cells from a single mouse. Measurements are representative of cell populations from experiments performed at least 2 times. Mean±SEM data is presented.

### DETAILED DESCRIPTION OF THE PREFERRED EMBODIMENTS

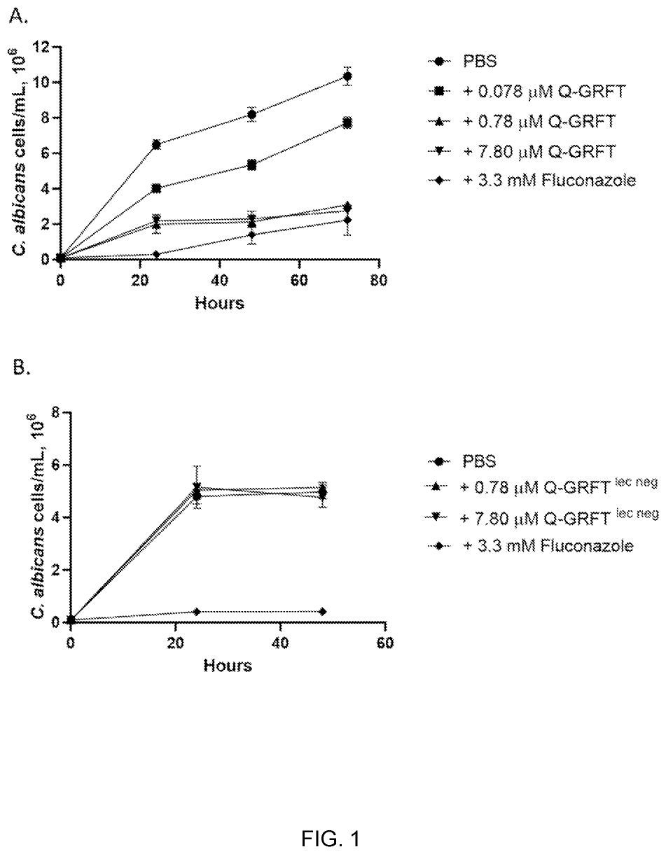

The inventors investigated the impact of Q-GRFT on the growth of Candida albicans ATCC 32032, as a representative of the gut fungal community. Yeast counts were determined using a Bio-Rad TC10™ Automated cell counter, Singapore, or an ECHO Rebel hybrid microscope (RBLTEW31), San Diego, USA. Following incubation with Candida albicans for 24, 48 and 72 hours (FIG. 1, panel A), Q-GRFT significantly inhibited fungal growth; p<0.0001 at all concentrations tested (7.80 μM (10.0 mg/mL), 0.78 μM (1.0 mg/mL) and 0.078 μM (0.1 mg/mL)). Q-GRFT possesses growth inhibitory activity as shown by incubating C. albicans with a Q-GRFT variant, Q-GRFT^{lec neg}, devoid of its glycan binding ability. Incubation of C. albicans with Q-GRFT^{lec neg} for up to 48 hours did not demonstrate any inhibitory impact on growth (FIG. 1, panel B), suggesting a role of Q-GRFT's binding on fungal inhibitory activity.

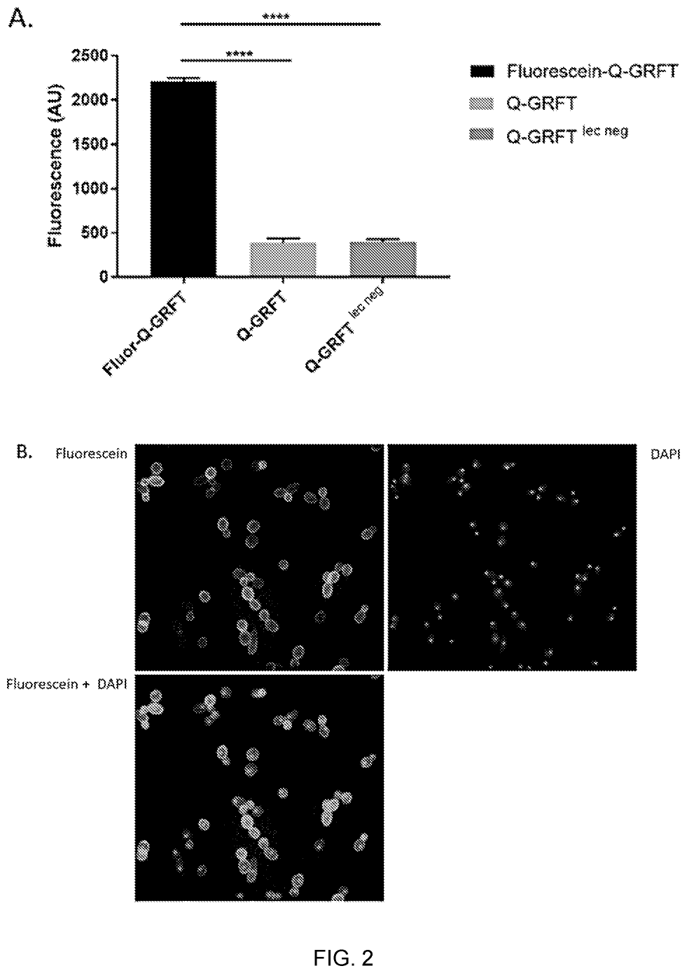

Since Q-GRFT inhibited the growth of C. albicans, the inventors sought to determine if this activity was dependent on Q-GRFT binding. C. albicans (1.0×10⁵ cells/mL) was cultured overnight with either Fluorescein-labeled Q-GRFT, unlabeled Q-GRFT or the Fluorescein-labeled Q-GRFT^{lec neg}. Cells were then centrifuged, washed, and fluorescence intensity of the pellet determined. C. albicans incubated with Fluorescein-Q-GRFT displayed the highest fluorescence intensity compared to the non-labeled Q-GRFT (p<0.0001) and Fluorescein-Q-GRFT^{lec neg} (p<0.0001) (FIG. 2, panel A). To confirm lectin binding, C. albicans was cultured overnight with either Fluorescein-Q-GRFT, Q-GRFT or Fluorescein-Q-GRFT^{lec neg}, and the cells visualized using fluorescence microscopy. Green fluorescence was observed around yeast cells incubated with Fluorescein-Q-GRFT, confirming Q-GRFT's binding to C. albicans (FIG. 2, panel B). No fluorescence was observed following incubation with Fluorescein-Q-GRFT^{lec neg} (data not shown).

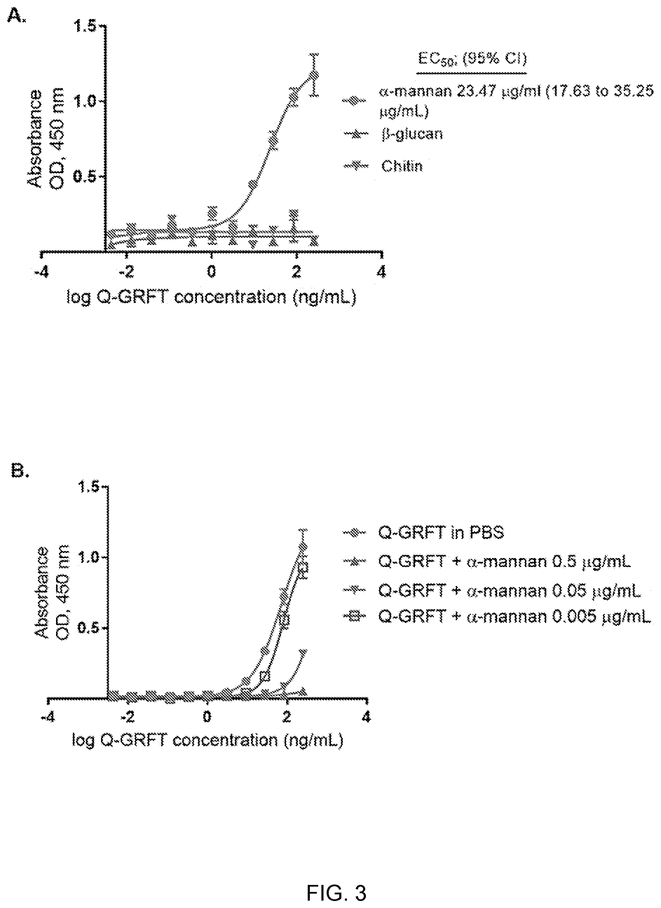

The inventors next investigated the C. albicans cell wall components to which Q-GRFT binds. Since the lectin bound to C. albicans, the inventors hypothesized that Q-GRFT likely binds to either chitin, glucans or mannans, which are predominant core components of the fungal cell wall. Using ELISA binding assays (FIG. 3), the inventors determined the ability of Q-GRFT to bind to α-mannan, β-glucan and chitin. Q-GRFT bound to α-mannan with EC₅₀ 23.47 μg/mL (95% CI 17.63 to 35.25 μg/mL). Q-GRFT did not bind to β-glucan or chitin. Because Q-GRFT is also being developed as a rectal microbicide for prevention of HIV infection, we sought to determine if the lectin's binding to gp120, a protein in the HIV envelope is inhibited in the presence of α-mannan. In a competition assay (FIG. 3, panel B), Q-GRFT's binding to gp120 was inhibited in the presence of high concentrations of α-mannan. The EC₅₀ in PBS was 72.46 μg/mL (95% CI 55.17 to 115.1 μg/mL), while in α-mannan 0.005 g/mL was 79.35 μg/mL (95% CI 69.19 to 97.85 μg/mL). Because Q-GRFT is known to bind gp120, its failure to bind gp120 in the presence of α-mannan is highly suggestive that the recombinant protein is binding α-mannan, not gp120, in this experiment.

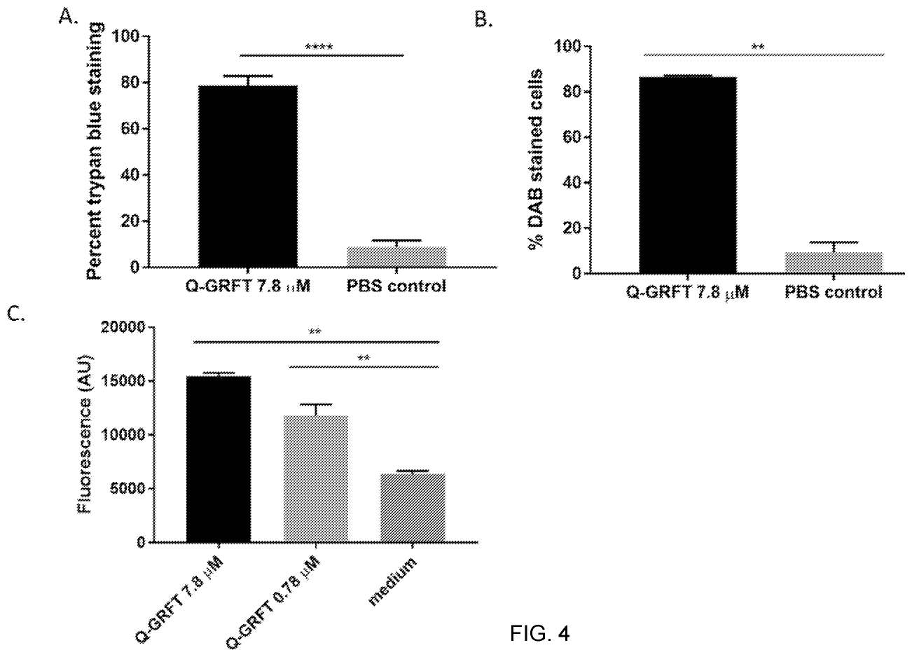

Treatment of C. albicans with helianthus annus (Helja) lectin has been shown to alter membrane permeability and induce intracellular formation of oxidative species. Therefore, the inventors hypothesized that Q-GRFT lectin may act similarly, altering fungal cell wall permeability and inducing expression of intracellular reactive oxidative species (ROS) that may result in cellular injury and/or damage leading to cell death. To demonstrate the impact on cell wall integrity, C. albicans was incubated with 7.8 μM Q-GRFT overnight, followed by Trypan Blue staining, with the dye uptake demonstrating breached cell wall and/or membrane integrity and penetration into dead and/or non-viable cells. Compared to the PBS vehicle, Q-GRFT treatment resulted in significant intracellular blue color staining, indicative of non-viable cells with impaired cell wall integrity, p<0.0001 (FIG. 4, panel A). To investigate the induction of ROS by Q-GRFT, Candida albicans cells were

incubated overnight with 7.8 M Q-GRFT followed by incubation with 3, 3-diaminobenzidine (DAB) for 2 hours. In the presence of peroxides, DAB is oxidized to an insoluble brown precipitate that is visualized within cells using optical microscopy. Compared to the PBS vehicle treated control, a significantly large proportion of Candida albicans cells incubated with Q-GRFT developed a brown intracellular precipitate. Quantification of this effect revealed that the presence of peroxides was significantly higher (p<0.0017) following Q-GRFT treatment than with PBS control (FIG. 4, panel B). To confirm the presence of ROS, the H2DCF-DA assay technique was used to profile the oxidative status of Q-GRFT-treated and PBS vehicle-treated control cells. This assay is dependent on cellular esterase ability to cleave acetate groups on H2DCF-DA, releasing an intermediate H₂DCF product which reacts with ROS forming fluorescent 2′,7′-dichlorofluorescein (DCF). Compared with PBS vehicle-treated control cells, Q-GRFT treatment was associated with higher fluorescence activity (p<0.002, p<0.006 for 7.8 μM and 0.78 μM respectively) following H₂DCF assay (FIG. 4, panel C). Centrifugation of cells during preparation for H₂DCF assay may induce ROS accounting for the low-level induction observed in the negative control (PBS vehicle-treated) cells. It is likely that different Q-GRFT concentrations elicit various levels of cellular stress, accounting for the differences in ROS formation for both 7.8 μM and 0.78 μM Q-GRFT treatments.

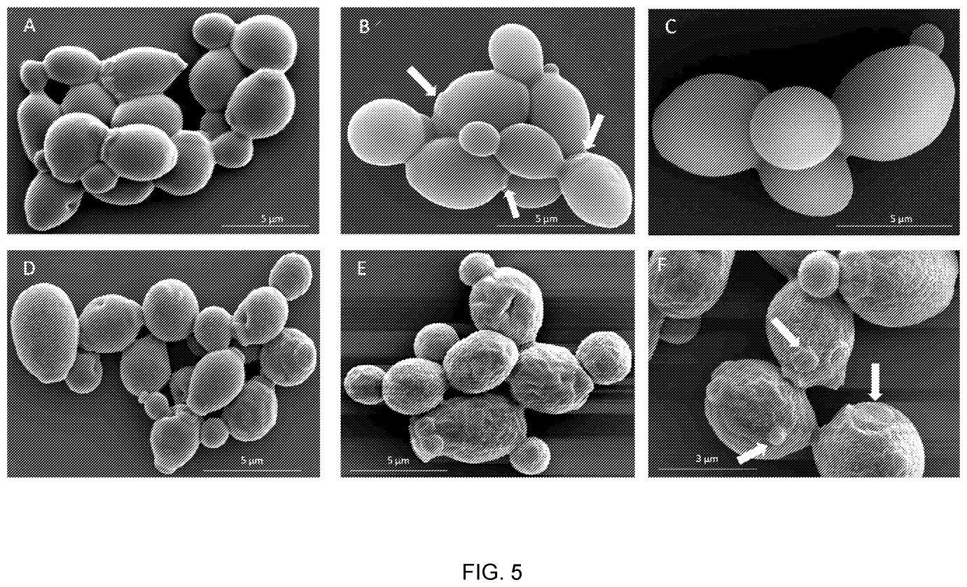

To further evaluate any structural changes to C. albicans following treatment with Q-GRFT, high resolution scanning electron microscopy (SEM) was performed for both Q-GRFT-treated and PBS vehicle-treated control cells (FIG. 5). Yeast cells were treated with either 7.8 μM Q-GRFT or PBS vehicle and incubated overnight at 37° C. prior to imaging. The PBS vehicle-treated control cells demonstrated a normal budding pattern, were predominantly spherical to oval in shape (FIG. 5, panel A), with polar buds and bud scars (FIG. 5, panel B) and smooth edges and surfaces (FIG. 5, panel C). Q-GRFT treated cells were spherical to circular (FIG. 5, panels D and E), rough in appearance, demonstrated desiccated and wrinkled surfaces with uniform indentations (FIG. 5, panels E and F), and with a loss of polar budding (FIG. 5, panel F). The rough and wrinkled appearance with loss of polar budding denotes dead cells unfit for further asexual reproduction.

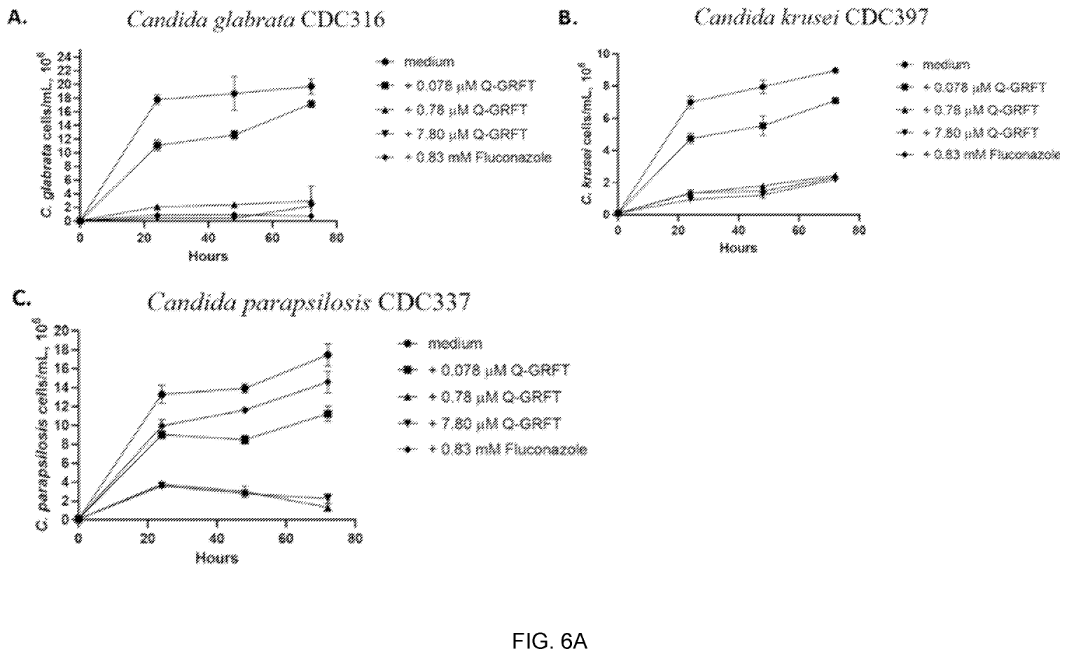

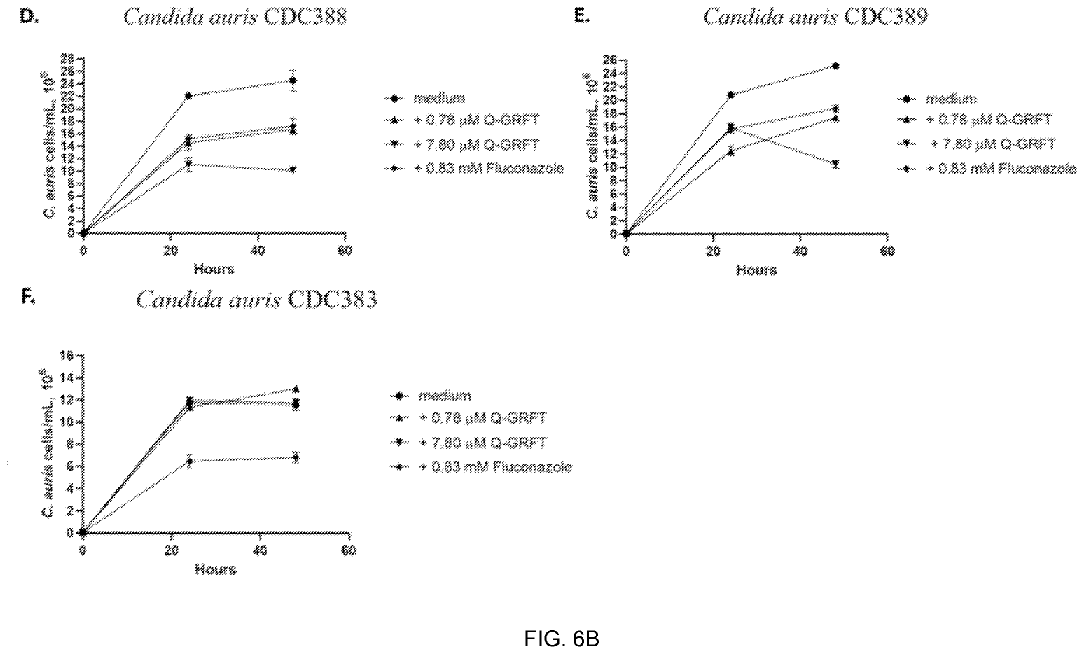

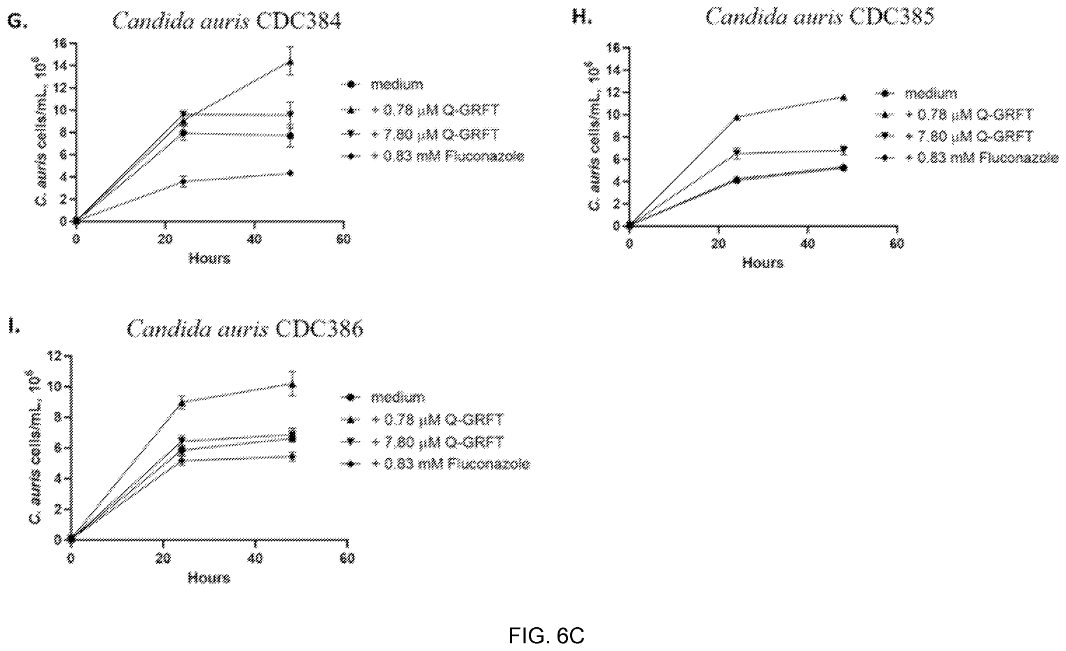

Given the recent increase in Candida species resistance to antifungal drugs, the impact of Q-GRFT on the growth of human pathogenic non-Candida albicans species including the multidrug resistant (MDR) Candida auris was investigated. Q-GRFT was incubated with Candida glabrata, Candida krusei and Candida parapsilosis all at a concentration of 1.0×10⁵ cell/mL were incubated with different concentrations of Q-GRFT and fluconazole control at 37° C. in Sabourand Dextrose culture media and growth monitored up to 72 hours. Fungal counts were performed using either a Bio-Rad TC10™ Automated cell counter, Singapore, or an ECHO Rebel hybrid microscope (RBLTEW31), San Diego, USA. Data represents mean±SD from 3 independent experiments. Compared to the medium control, Q-GRFT significantly inhibited the growth of all the species tested, with the greatest effect demonstrated with the 7.80 μM lectin concentration, p<0.0001 for all species and concentrations tested (FIG. 6A, panels A, B and C). When incubated with Candida auris, Q-GRFT significantly inhibited the growth of strains Candida auris CDC388 and Candida auris CDC389, p<0.0001, for the lectin concentrations 0.78 μM and 7.80 μM tested (FIG. 6B, panels D and E). There was no observable impact on the growth of strains Candida auris CDC383 (FIG. 6B, panel F), Candida auris CDC384 (FIG. 6C, panel G), Candida auris CDC385 (FIG. 6C, panel H) and Candida auris CDC386 (FIG. 6C, panel I).

Starting with a maximum concentration of 95 μg/mL, minimum inhibitory concentrations (MICs) were determined for Q-GRFT's activity against different Candida isolates and are summarized in Table 1. The MIC₉₀s of Q-GRFT for C. albicans, C. glabrata, C. parapsilosis, C. krusei, C. auris CDC388 and C. auris CDC389 were 6, 95, 24, 95, 48 and 95 μg/mL, respectively, while MIC₉₀s for C. albicans, C. parapsilosis and C. auris CDC389 were 95 μg/mL for all isolates, respectively. In some embodiments, methods for treatment of fungal infections, including candidiasis, include topical application of a composition including Q-GRFT at a concentration of about 1% by weight, a non-zero concentration of not more than 1% by weight, a concentration of 0.001% to 0.5% by weight, a concentration of 0.01% to 0.3% by weight, a concentration of at least 1 μg/mL, a concentration between 1 μg/mL and 10 mg/mL (7.8 μM), a non-zero concentration of less than 10 mg/mL (7.8 μM), or a concentration between 6 μg/mL and 95 μg/mL at least twice daily for 3-10 days, 4-8 days, 5-7 days, or until the fungal infection is no longer detectable. The composition is formulated as a gel or liquid and administered at a volume of 0.1 mL to 10 mL, 0.1 mL to 5.0 mL, 0.1 mL to 2.0 mL or 0.5 mL to 1.0 mL per application.

TABLE 1
MIC₅₀s and MIC₉₀s of Q-GRFT against different Candida species

| Candida isolate | MIC₅₀ (μg/mL) | MIC₉₀ (μg/mL) |
|---|---|---|
| Candida albicans ATCC32020 | 6 | 95 |
| Candida glabrata CDC316 | 95 | — |
| Candida parapsilosis CDC337 | 24 | 95 |
| Candida krusei CDC397 | 95 | — |
| Candida auris CDC388 | 48 | 95 |
| Candida auris CDC389 | 95 | — |

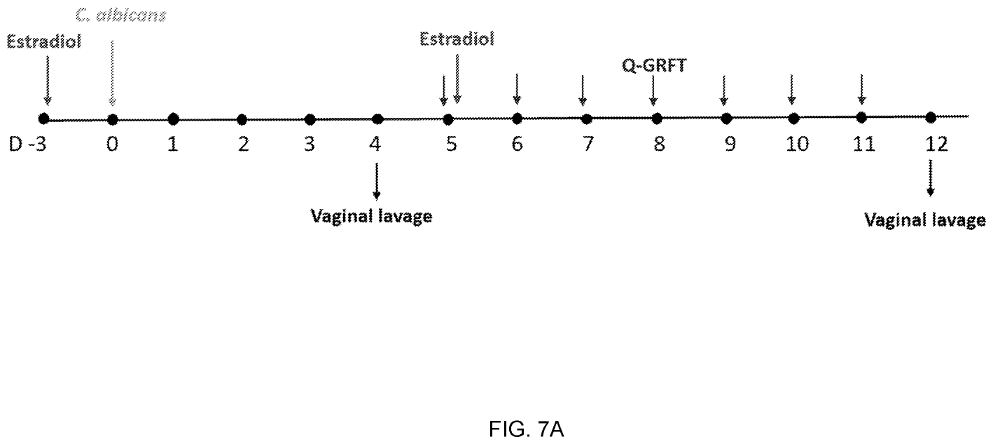

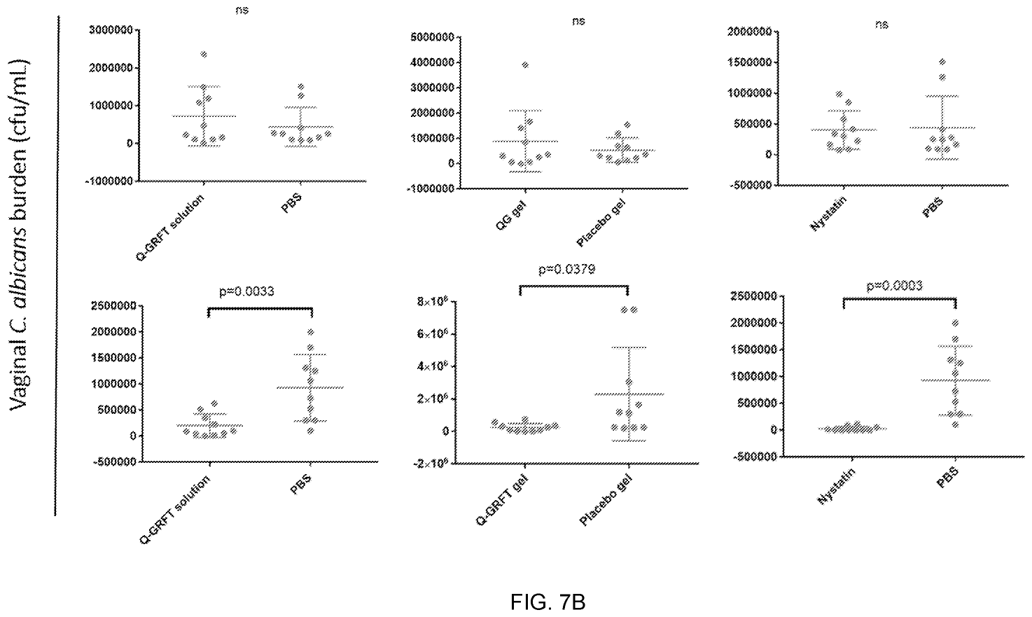

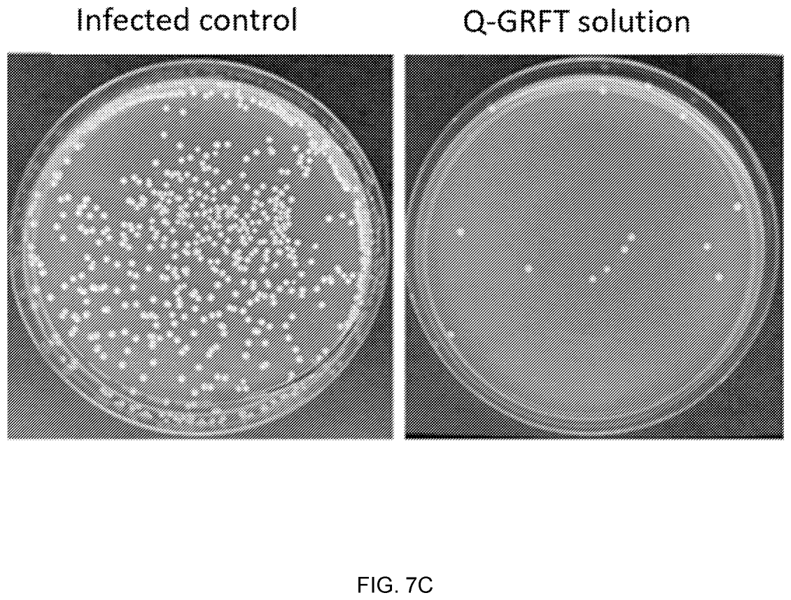

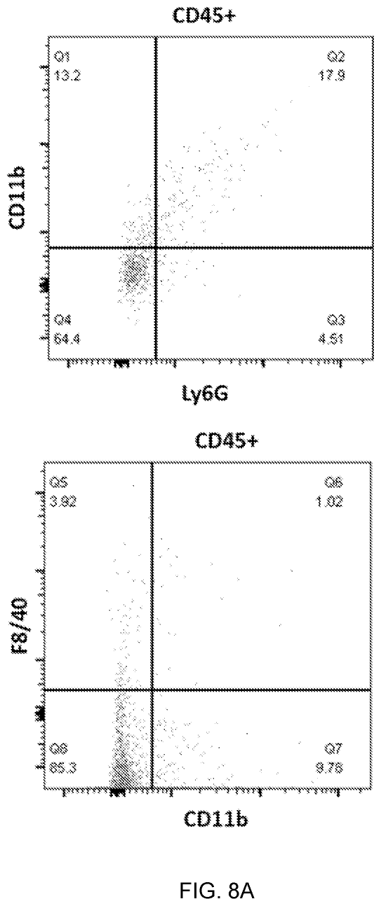

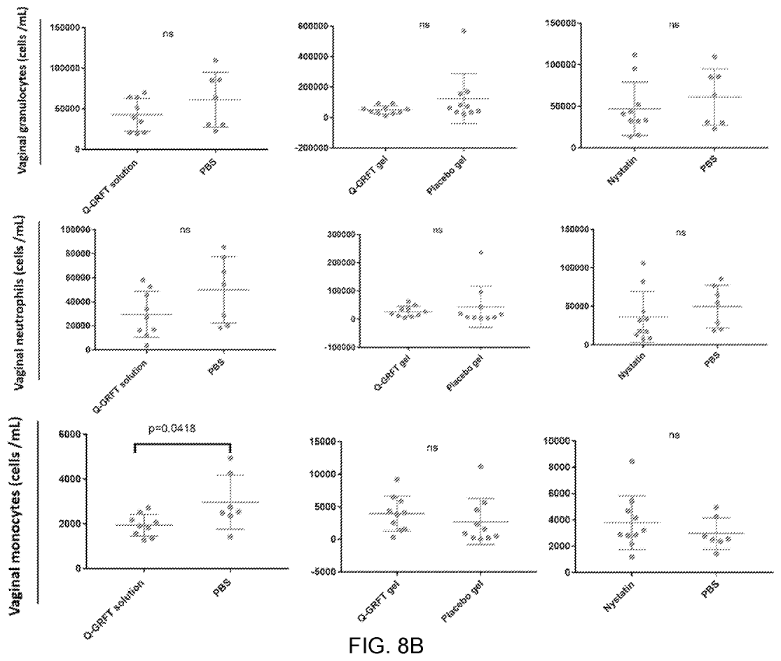

FIGS. 7A, 7B, and C illustrate that application of Q-GRFT solution and gel result in lower fungal burden as compared with control treatments, preventing vaginal candidiasis in a first therapeutic murine model. FIGS. 8A and 8B indicate that Q-GRFT does not induce overt changes in vaginal innate immune response to candidiasis as treatment with Q-GRFT solution and gel did not result in significant observable changes in the population of vaginal granulocytes and neutrophils. Monocyte population was modestly lower following treatment with Q-GRFT solution.

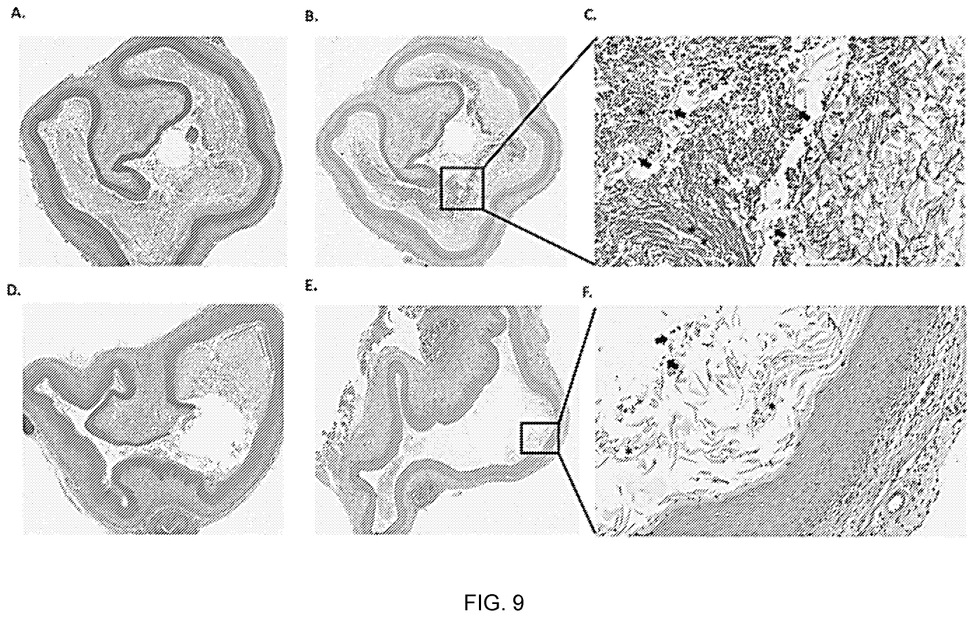

To further investigate the effect of Q-GRFT on vaginal candidiasis, the impact of topical administration on the histology of infected tissues was evaluated at the end of the drug treatment period. FIG. 9 displays histology slides, wherein C. albicans are indicated by asterisks “*” and neutrophils are indicated by arrows. Microscopic analysis revealed that infected untreated animals displayed significant vaginal luminal congestion with high fungal growth/burden (FIG. 9, panels A, B, and C), unlike Q-GRFT-treated animals that displayed lower congestion (FIG. 9, panels D, E, and F). Q-GRFT is associated with a lower C. albicans vaginal burden, with a visibly distinct decrease in fungal growth in the vaginal lumen in Q-GRFT-treated samples as compared to PBS-treated samples. Consistent with microbiological observations, H&E and PAS staining demonstrated that Q-GRFT was an effective treatment against treated vaginal candidiasis.

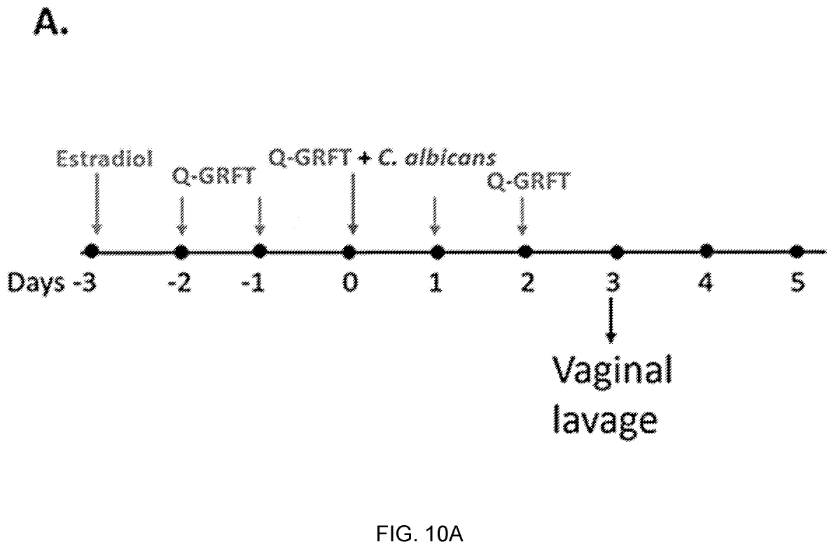

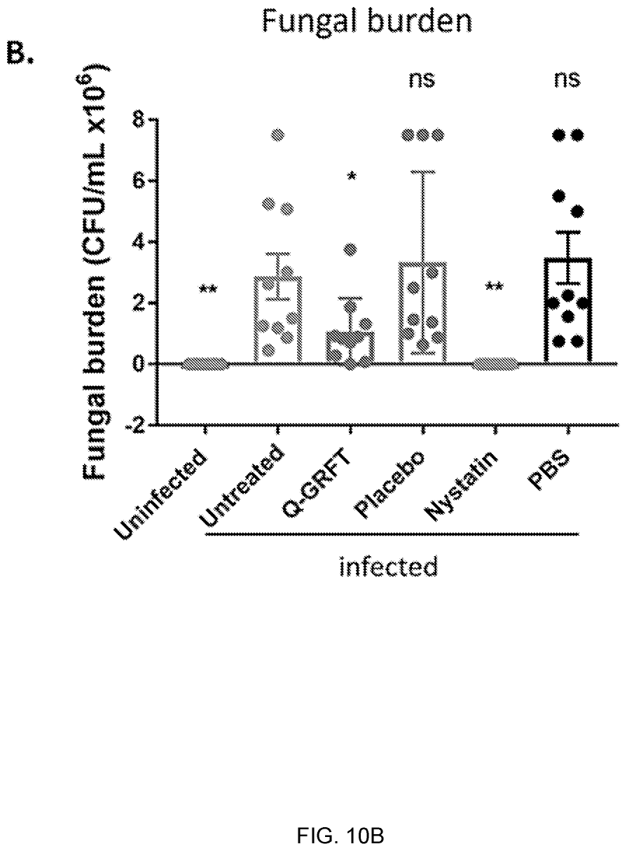

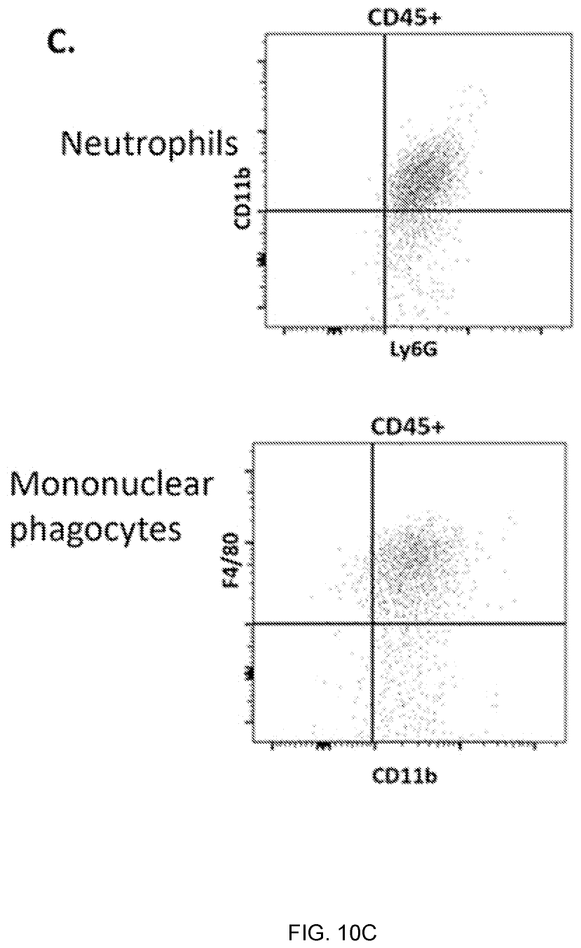

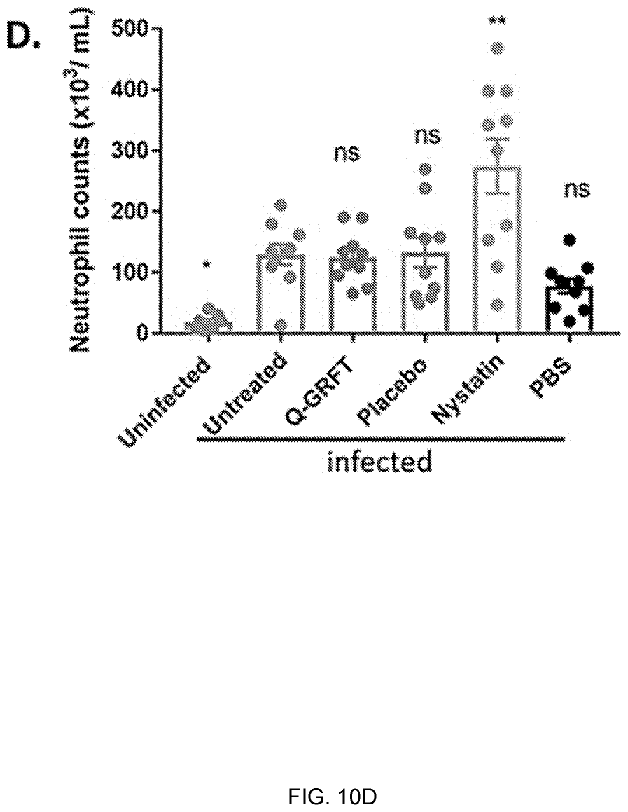

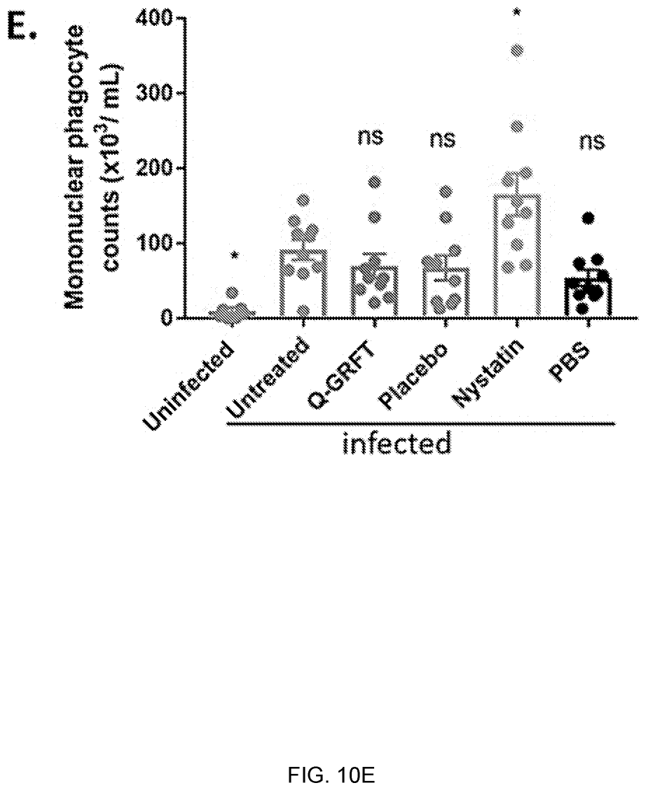

To evaluate the efficacy of Q-GRFT in a preventive murine model, an experimental model for vaginal infection was established as shown in FIG. 10A. Female CBA/J mice were injected subcutaneously with estradiol, followed by twice daily vaginal instillation of a carbomer gel formulation (such as, for example, Carbopol®). The carbomer gel formulation delivered 400 ng Q-GRFT per dose twice daily for 5 days. The animals were challenged with C. albicans on day 3. Vaginal lavage was performed 24 hours following administration of the final Q-GRFT treatment. The efficacy of Q-GRFT in the prevention of vaginal candidiasis was determined by establishing the fungal burden in vaginal lavage fluids after vaginal pre-treatment, fungal inoculation, and follow-up treatment with Q-GRFT. Fungal burden was evaluated by plating lavage fluids on Sabourand agar plates that were incubated for 48 hours at 30° C., followed by counting of colonies. Q-GRFT treatment resulted in a significantly lower fungal burden when compared with the infected untreated controls (P=0.0417) (FIG. 10B). Similarly, treatment with the positive control nystatin, a polyene antifungal agent, resulted in a significantly lower fungal burden (P=0.0016), while there was no inhibition demonstrated with PBS (P=0.4849) and placebo (P=0.5963) when compared with the infected controls. Additionally, uninfected animals did not demonstrate any fungal growth (P=0.0016). Upon infection and epithelial penetration, tissue resident-macrophages are

among the initial immune cells that encounter Candida, and phagocytose the fungal cells to achieve clearance. Furthermore, pro-inflammatory cytokines released by macrophages and epithelial cells recruit neutrophils and inflammatory monocytes to eradicate Candida infection. However, recent findings have suggested that while polymorphonuclear neutrophils (PMNs) are generally protective against C. albicans at other body sites, they do not appear to be protective in the vagina. Depletion of PMNs using an anti-Ly6G antibody was shown not to impact fungal burden.

Therefore, using flow cytometry, the inventors sought to determine if pre-treatment with Q-GRFT influenced the expression of vaginal innate immune cells [neutrophils (CD45⁺,Ly6G⁺,CD11b⁺) (FIG. 10C, top panel) and mononuclear phagocytes (CD45⁺CD11⁺F4/80⁺) (FIG. 10C, bottom panel) in vaginal infection. Compared to infected controls, there was no difference in the population of neutrophils following pre-treatment with either Q-GRFT (P>0.9999), placebo (P>0.9999), or PBS (P>0.6510) (FIG. 1D). This is consistent with reported observations that PMNs do not contribute to clearance during VVC. In addition, treatment with nystatin resulted in significantly higher populations of neutrophils (P=0.0011), while uninfected animals had lower neutrophils (P=0.0483), in comparison with infected controls. Similarly, there was no difference in mononuclear phagocyte populations following treatment with Q-GRFT (P=0.9461), placebo (P=0.9155), and PBS (P=0.6263), in comparison with the infected controls. Nystatin treated animals were associated with higher monocyte populations (P=0.0380), while uninfected animals demonstrated significantly lower monocytes (P=0.0368) than infected animals. These results demonstrate that while Q-GRFT significantly inhibits Candida growth in a preventative murine model, this effect is likely independent of the inflammatory immune response.

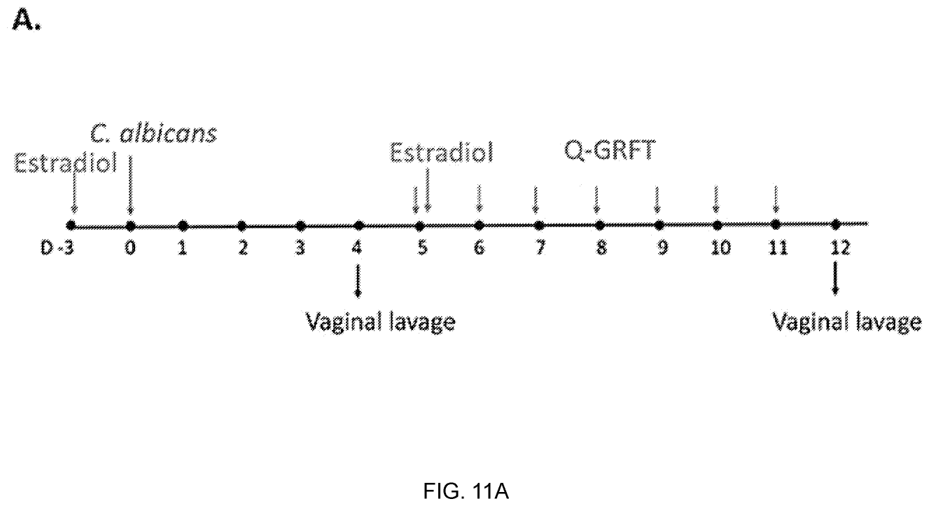

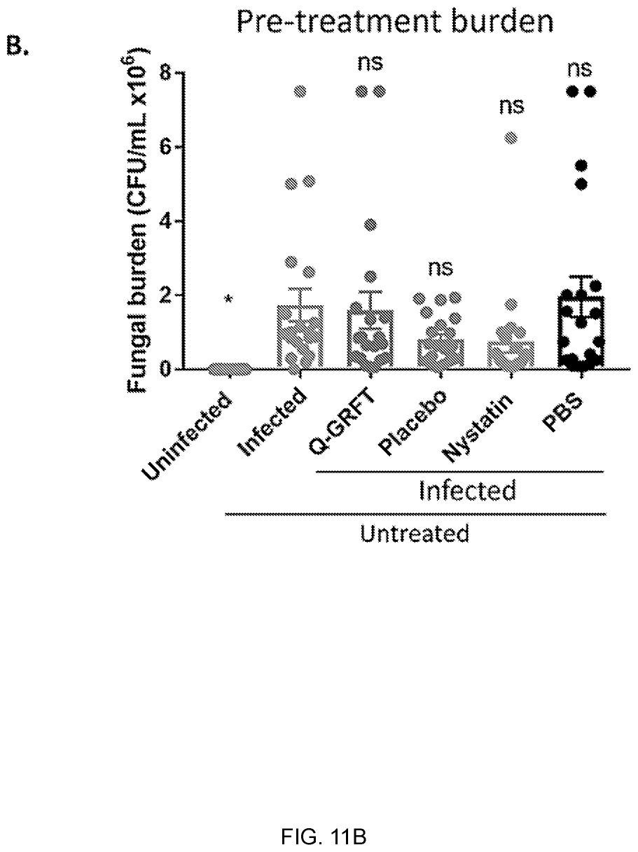

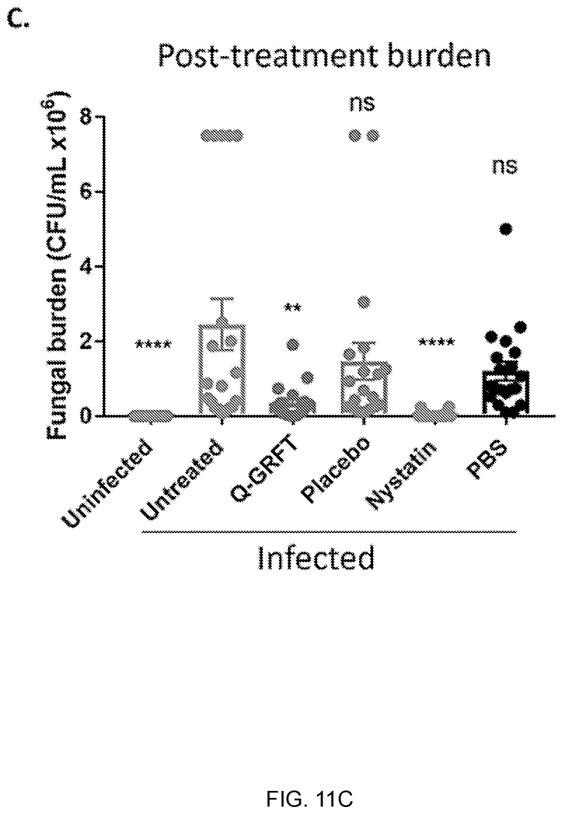

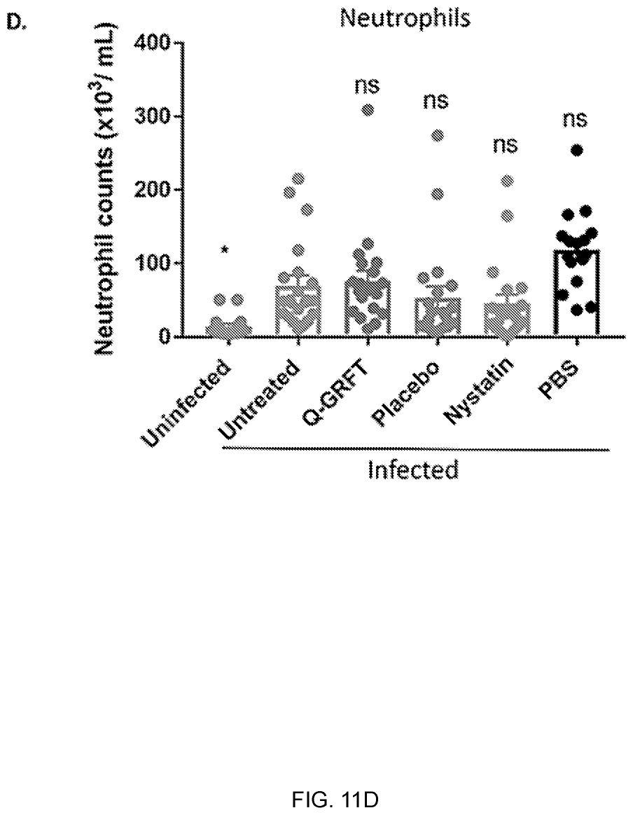

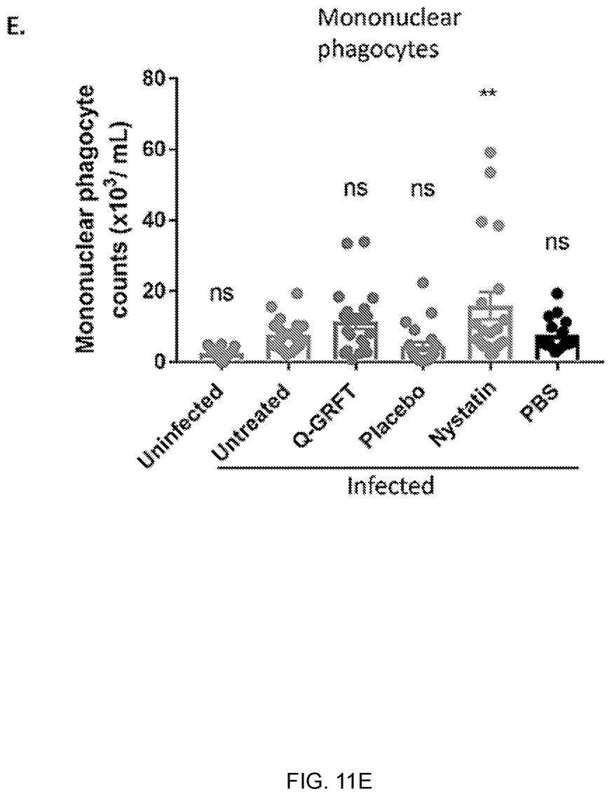

To study the role of Q-GRFT in the treatment of candidiasis, a murine therapeutic experimental model was developed as shown in FIG. 11A. Mice were injected subcutaneously with estradiol, followed by inoculation with C. albicans vaginally 3 days later. Vaginal lavage was performed on day 4 following fungal challenge, to determine baseline fungal burden. Twice daily vaginal instillation of 400 ng Q-GRFT was started on day 5 and continued for a total of 7 days. A vaginal lavage was performed 24 hours after the final dose to determine fungal burden by colony counts on Sabourand agar plates, and immune response to treatment using flow cytometry. Pre-treatment fungal burden (FIG. 11B) confirmed that all mice had established vaginal infection prior to initiating treatment, with no significant differences in fungal burden in any of the infected groups prior to initiation of treatment. Compared to placebo, treatment with topical vaginal Q-GRFT gel resulted in a significant inhibition of C. albicans burden (P=0.0379), similar to that seen with the control nystatin (P=0.0003), at the end of the dosing period (FIG. 11C). These results indicated that Q-GRFT was an effective treatment for vaginal candidiasis in a murine model.

Pro-inflammatory cytokines released by macrophages and epithelial cells recruit neutrophils and inflammatory monocytes during Candida infection. Therefore, we next sought to determine if treatment with Q-GRFT influenced the expression of vaginal innate immune cells, neutrophils (CD45⁺Ly6G⁺CD11b⁺), (FIG. 11D), and mononuclear phagocytes (CD45⁺CD11b⁺F4/80⁺), (FIG. 2E), in candidiasis using flow cytometry. Compared to infected controls, Q-GRFT did not induce any significant changes in populations of both neutrophils, P=0.7279, and mononuclear phagocytes, P=0.1960. Similarly, neutrophils populations were not significantly different between infected controls and nystatin treated mice, P=0.1771, while monocytes were elevated following treatment, P=0.0055. Compared with the infected untreated controls, uninfected mice demonstrated significantly lower neutrophils (P=0.0039), but not mononuclear phagocytes, P=0.0873. Both placebo and PBS did not result in any changes in neutrophil (P=0.3626, P=0.111), and monocyte (P=0.2464, P=0.9939) populations, respectively, when compared to infected untreated animals. These results demonstrate that Q-GRFT does not induce overt changes in neutrophil and mononuclear phagocytic populations following vaginal infection with C. albicans.

With respect to the aforementioned murine models, the C. albicans ATCC 32032 strain was grown on Sabourand dextrose agar plates overnight at 30° C. prior to use. 10 milliliters of Sabourand dextrose media were inoculated with 1 colony of C. albicans from the agar plate and incubated at 30° C. with shaking for 18 hours. Cells were then sub-cultured 1:100 dilution overnight, followed by preparation of 1.0×10⁸ cells/mL blastospores from the stationary phase, that were suspended in sterile PBS. Cells were kept on ice until when vaginal inoculation was performed in mice. Twenty microliters of the C. albicans preparation were dispensed into each mouse's vagina using a P50 positive displacement pipettor.

In summary, Q-GRFT (i) inhibits vaginal C. albicans growth in an in vivo murine model, (ii) is associated with a lower fungal burden in vaginal candidiasis, and (iii) may not induce overt changes to vaginal innate immune response to candidiasis. This disclosure should not be read as limiting the use of Q-GRFT as an anti-fungal composition only for vaginal candidiasis. C. albicans is found in the mycobiome in the gut and colon, and Q-GRFT may function as an effective anti-fungal composition in the gut and colon, skin, and other areas of the body, including systemic infections, and against other fungal infections. Q-GRFT binds to α-mannan in the fungal cell wall and inhibited the growth of C. albicans. Antifungal activity is dependent on lectin binding since Q-GRFT^{lec neg} neither bound nor inhibited C. albicans growth.

Estradiol (SIGMA Life Science, Lot# BCBW5905) was dissolved in sesame oil (SIGMA, Lot# MKCG9353) to a concentration of 0.5 mg/mL. Mice were then injected subcutaneously with 100 μL of the hormonal preparation in the lower abdomen 3 days prior to C. albicans challenge, and then once weekly for the duration of the experiment. To perform the lavage, 100 μL of sterile PBS were dispensed into the mouse vagina and aspirated back and forth several times, and then transferred to labelled Eppendorf tubes on ice. The lavage was then diluted 1:100, and 50 μL of the diluted fluid plated on Sabourand agar. Colli rollers were used to spread the lavage. The plates were incubated at 30° C. for 24-48 hours, and colonies counted to establish the fungal burden.

For the vaginal treatment, forty microliters (40 μL) of a 1% Q-GRFT gel formulated in carbomer (400 ng), 40 μL of carbomer placebo gel, 100 μL of nystatin solution at a concentration of 20 mg/mL (Mayne Pharma, Greenville, NC, USA) and 100 μL of sterile 1X PBS were instilled per vaginum in mice from the different animal groups, using appropriate pipettors. While Q-GRFT is discussed herein as an anti-fungal agent using a carbomer carrier, other biologically compatible suspending agents, gel bases, emulsifiers, and binding agents as known in the art may also be used as carriers.

Q-GRFT exposure to C. albicans may result in an osmotic imbalance caused by lectin-mediated disruption in cell wall integrity. Q-GRFT induced marked alterations in the physical appearance of C. albicans with a marked shriveled appearance and collapsed cells with surface indentations. Q-GRFT-treated cells also demonstrate an increased attempt in budding with multiple bud scars and a loss of the normal polar budding orientation. This is indicative that Q-GRFT-induced changes affect normal cell division. The loss of polar budding and multiple bud scars may be an attempt by C. albicans to divide multiple times to escape stress-induced conditions. This failed attempt is likely compounded by the down regulation of Tos4 gene, observed following Q-GRFT treatment. Tos4 gene regulates the G1/S cell cycle phase, promoting cell division. This downregulation, together with other intracellular injury processes, result in failure to complete cell division and subsequently lead to cell death. Additionally, in response to oxidative stress, C. albicans expresses antioxidant genes to neutralize and escape stress, including superoxide dismutase (SOD), glutathione peroxidase (GPX2), thioredoxin (TRX) and thioredoxin reductase (TRR). Q-GRFT treatment was associated with up-regulation of SOD while cells treated with the non-binding Q-GRFT^{lec neg} exhibited downregulation of GPX2 and TRR1. There is a likelihood for cells incubated with Q-GRFT^{lec neg} to undergo stressful growth conditions given volume and space limitations with this assay. When cells grow uninhibited in media, they will reach a critical mass when they start to compete for nutrients within the restricted space. This has the potential to induce metabolic responses within cells to escape these stressful conditions. However, given the clear differential expression of genes following Q-GRFT treatment in comparison to Q-GRFT^{lec neg} treated cells, it is evident that the lectin does impact multiple metabolic pathways within C. albicans after treatment. Cell cycle arrest, disaccharide metabolism, biofilm formation and DNA strand elongation were among the upregulated pathways following Q-GRFT lec neg treatment. Similarly, QG treatment demonstrated upregulated stress response pathways including monosaccharides, glucose, galactose and amino acid metabolism, biofilm formation and DNA replication, among others.

To date, there is no demonstrated toxicity, T-cell activation, or immunological stimulation of GRFT or Q-GRFT in in vitro and in vivo studies. Cytokines and chemokines secretion by epithelial cells is only minimally changed upon treatment with GRFT. The lack of difference in populations of neutrophils and monocytes triggered following vaginal infection in both Q-GRFT treated and untreated animals indicate a direct inhibitory role of the lectin against Candida. This disclosure demonstrates that Q-GRFT significantly inhibited infection in a preventive model, and enhanced candidiasis clearance in murine therapeutic studies. Altogether, these data suggest that Q-GRFT likely directly inhibits vaginal Candida growth, regardless of the inflammatory status in the local milieu.

In addition to Candida albicans, Q-GRFT demonstrates potent inhibitory activity against other Candida species of clinical importance, such as Candida glabrata, Candida krusei and Candida parapsilosis. Interestingly, C. krusei has been described to harbor innate resistance against fluconazole, while azole resistance is increasingly being documented for C. glabrata and C. parapsilosis. In addition, our study established that Q-GRFT demonstrates growth inhibition of Candida auris CDC388 and Candida auris CDC389 strains. However, growth in strains Candida auris CDC383, Candida auris CDC384, Candida auris CDC385 and Candida auris CDC386 was not impacted following incubation with Q-GRFT. C. auris exhibits multi drug resistance, and pan-resistant strains have recently been identified. Overall, these findings suggest that Q-GRFT's anti-Candida activity may be beneficial as an additional strategy or alternative to traditional antifungal treatment, given the inhibitory activity observed even in strains known to demonstrate resistance to common antifungal agents.

The foregoing detailed description is given primarily for clearness of understanding and no unnecessary limitations are to be understood therefrom for modifications can be made by those skilled in the art upon reading this disclosure and may be made without departing from the spirit of the invention.

1. A method for preventing or treating fungal infections, comprising applying a composition including griffithsin protein to a subject.

2. The method of claim 1, wherein the fungal infection is candidiasis.

3. The method of claim 1, wherein the fungal infection is vaginal candidiasis and the subject is a subject vagina.

4. The method of claim 1, wherein the griffithsin protein is a mutant griffithsin protein.

5. The method of claim 4, wherein the mutant griffithsin protein is Q-GRFT.

6. The method of claim 1, wherein the composition includes griffithsin protein at a concentration effective to reduce fungal infection.

7. The method of claim 1, wherein the composition includes griffithsin protein at a non-zero concentration less than 1% by weight.

8. The method of claim 7, wherein the composition includes griffithsin protein at a concentration within the range of 0.001% to 0.5% by weight.

9. The method of claim 8, wherein the composition includes griffithsin protein at a concentration within the range of 0.01% to 0.3% by weight.

10. The method of claim 1, wherein applying the composition to the subject includes applying the composition topically to a subject vagina.

11. The method of claim 1, wherein applying the composition includes applying the composition at least twice a day for at least three days.

12. The method of claim 1, wherein the composition further includes a carrier.

13. The method of claim 12, wherein the carrier is a biologically-compatible suspending agent.

14. The method of claim 12, wherein the carrier is a carbomer gel.

15. An antifungal composition comprising a griffithsin protein and a carrier, wherein the griffithsin protein is present at a concentration effective to reduce fungal infection.

16. The composition of claim 10, wherein the griffithsin protein is a mutant griffithsin protein.

17. The composition of claim 16, wherein the mutant griffithsin protein is Q-GRFT.

18. The composition of claim 15, wherein the fungal infection is candidiasis.

19. The composition of claim 15, wherein the carrier is a biologically-compatible suspending agent.

20. The composition of claim 19, wherein the carrier is a carbomer gel.

21. The composition of claim 15, wherein the concentration is a non-zero concentration less than 1% by weight.

22. The method of claim 21, wherein the concentration is within the range of 0.001% to 0.5% by weight.

23. The method of claim 8, wherein the concentration within the range of 0.01% to 0.3% by weight.


SEQUENCE LISTING (2 sequences)

SEQ ID NO: 1 - protein, 121 aa, Griffitsia sp.
SLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 2 - protein, 121 aa, synthetic construct
SLTHRKFGGSGGSPFSGLSSIAVRSGSYLDAIIIDGVHHGGSGGNLSPTFTFGSGEYISNMTIRSGDYIDNISFETNQGRRFGPYGGSGGSANTLSNVKVIQINGSAGDYLDSLDIYYEQY